(19) United States
(12) Patent Application Publication
(10) Pub. No.: US 2023/0265056 A1
(43) Pub. Date: Aug. 24, 2023

(54) CRYSTALLINE FORMS OF BENZAMIDE COMPOUND AND PROCESS FOR PREPARING THE SAME

(71) Applicant: CRYSTAL PHARMATECH CO., LTD., Suzhou (CN)

(72) Inventors: Xia LU, Suzhou (CN); Xiaoyu ZHANG, Suzhou (CN); Ming MA, Suzhou (CN)

(73) Assignee: CRYSTAL PHARMATECH CO., LTD., Suzhou (CN)

(21) Appl. No.: 18/168,583

(22) Filed: Feb. 14, 2023

Related U.S. Application Data
(63) Continuation of application No. PCT/CN2021/112397, filed on Aug. 13, 2021.

(30) Foreign Application Priority Data
Aug. 14, 2020 (CN) 202010816335.6

(51) Int. Cl. C07D 239/47

(52) U.S. Cl. CPC C07D 239/47; C07B 2200/13

(57) ABSTRACT

The present disclosure relates to crystalline forms of benzamide compound and processes for preparing the same. Provided are crystalline Form J and an amorphous form of a compound of formula (I), a process for preparing the same, and use thereof. The crystalline Form J has the advantages in at least one of solubility, melting point, stability, dissolution, hygroscopicity, adhesion, fluidity, biological effectiveness, processing performance, purification, formulation production, safety, and the like, which provides a new and better choice for the preparation of pharmaceutical formulations containing the compound of formula (I), and has a very important significance for the drug development.

### CROSS-REFERENCE TO RELATED APPLICATIONS

This application is a Continuation of International Application No. PCT/CN2021/112397 filed on Aug. 13, 2021, which claims the priory of Chinese Patent Application No. 202010816335.6 filed on Aug. 14, 2020, the contents of which are hereby incorporated by reference.

### TECHNICAL FIELD

The present disclosure relates to the field of pharmaceutical chemistry, and in particular to crystalline forms of a benzamide compound and process for preparing the same.

### BACKGROUND

Bruton's tyrosine kinase (BTK) is a member of the cytosolic protein tyrosine kinase family, which selectively expresses in immune cells such as macrophages, mast cells, basophils, platelets and B cells, and plays an important role in development process of B cell. Accordingly, BTK-related gene variation are closely related to the occurrence and development of B-cell-associated autoimmune diseases, including Bruton's hypogammaglobulinemia, multiple sclerosis, asthma, atopic dermatitis, urticaria, chronic lymphocytic leukemia, mantle cell lymphoma and other tumors. Therefore, small molecule inhibitors targeting BTK has gained extensive attention in the field of pharmaceutical research.

N-(3-(6-amino-5-(2-(N-methylacrylamido)ethoxy)pyrimidin-4-yl)-5-fluoro-2-methylphenyl)-4-cyclopropyl-2-fluorobenzamide is an efficient and selective BTK inhibitor, which is useful for the treatment of chronic urticaria, asthma and Sjogren's syndrome. Its structural formula is as follows:

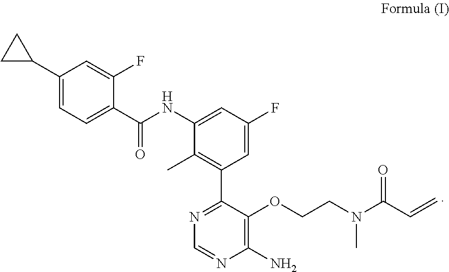 [SMILES: C=CC(=O)N(C)CCOc1c(N)ncnc1-c1cc(F)cc(NC(=O)c2ccc(C3CC3)cc2F)c1C]

WO2015079417A1 discloses the compound shown in formula (I) and a synthetic process therefor. According to the synthetic process, the final product was purified via column chromatography using a mixed solvent of dichloromethane, methanol and ammonium hydroxide. Considering the purification process is complicated and needs a huge volume of solvent, it is difficult to meet the requirement of energy consumption and environmental protection in industrial production. Compared to column chromatography, using crystallization method for purification may be more suitable in industrial production since it does not need special chromatographic equipment, shows a good connection between upstream and downstream processes, and consumes less solvent. WO2020234779A1 discloses anhydrous crystalline forms of the compound shown in formula (I), namely Form A, Form B and Form C. Form B and Form C are less thermodynamically stable, which would covert to Form A under certain conditions. Based on the properties of stability and hygroscopicity, Form A is preferred than other disclosed forms. The solubility of a drug in biological media is a key factor affecting the absorption and utilization of the drug in vivo. Different crystal forms show different solubility and thus different absorption and utilization in vivo. The development of crystal forms with better solubility is of great significance for improving the absorption and utilization of the drug.

In addition, different crystalline forms of the same drug have significant differences in solubility, melting point, density, stability, and the like, thus affecting stability, homogeneity, bioavailability, efficacy and safety of the drug to greater or lesser degrees. Therefore, it is one of the important research contents that cannot be ignored to comprehensively and systematically carry out polymorph screening and select the most suitable crystalline form in drug research and development.

### SUMMARY

The present disclosure provides a crystalline form (Form J) and amorphous form of Formula (I) and process for preparation the same.

1. A J-type crystalline form of a compound, represented by formula (I), N-(3-(6-amino-5(2-(N-methylacrylamido)ethoxy)pyrimidin-4-yl)-5-fluoro-2-methylphenyl)-4-cyclopropyl-2-fluorobenzamide, i.e., crystalline Form J, wherein, by using the Cu—Kα radiation, crystalline Form J has an X-ray powder diffraction pattern comprising characteristic peaks at the 2θ values of 6.0°±0.2°, 6.8°±0.2°, and 20.6°±0.2°.

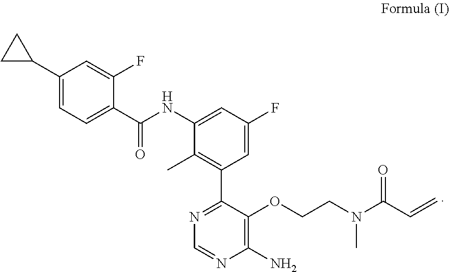 [SMILES: C=CC(=O)N(C)CCOc1c(N)ncnc1-c1cc(F)cc(NC(=O)c2ccc(C3CC3)cc2F)c1C]

In an embodiment of the present disclosure, crystalline Form J according to the above-mentioned item 1, has an X-ray powder diffraction pattern comprising characteristic peak(s) at one, two or three 2θ values of 12.2°±0.2°, 13.7°±0.2°, and 25.8°±0.2°.

3. In an embodiment of the present disclosure, crystalline Form J according to the above-mentioned item 1 or 2, has an X-ray powder diffraction pattern comprising characteristic peak(s) at one, two or three 2θ values of 12.2°±0.2°, 13.7°±0.2°, and 25.8°±0.2°.

4. The process for preparing Form J according to any of the above-mentioned items 1 to 3, comprising:

dissolving the compound represented by formula (I) in an organic solvent; and obtaining the crystalline Form J by evaporating a filtrate obtained after filtration.

5. A pharmaceutical composition, containing the crystalline Form J according to any one of the above-mentioned items 1 to 3 and a pharmaceutically acceptable carrier.

6. A pharmaceutical composition having BTK inhibitory activity, containing the crystalline Form J according to any one of the above-mentioned items 1 to 3 as an active component.

7. A drug for preventing or treating chronic urticaria, asthma and Sjogren's syndrome, comprising the crystalline Form J according to any one of the above-mentioned items 1 to 3 as an active component.

According to the present disclosure, the compound of formula (I) as raw material refers to its solid (crystal or amorphous), semi-solid, wax or oil form. In some embodiments, the compound of formula (I) as raw material is in the form of solid powder. The “stirring” is completed by conventional methods in the art, such as magnetic stirring or mechanical stirring, and the stirring speed is 50-1800 rpm. In some embodiments, the stirring speed for the magnetic stirring is 300-900 rpm, and the stirring speed for the mechanical stirring is 100-300 rpm.

In the present disclosure, “crystal” or “crystalline form” refers to the crystal or the crystalline form being identified by the X-ray diffraction pattern shown herein. Those skilled in the art are able to understand that physicochemical properties discussed herein can be characterized. The experimental errors depend on the instrument conditions, the sampling processes and the purity of samples. In particular, those skilled in the art generally know that the X-ray diffraction pattern typically varies with the experimental conditions. In particular, it is necessary to point out that, the relative intensity of the diffraction peaks in the X-ray diffraction pattern may also vary with the experimental conditions; therefore, the order of the diffraction peak intensities cannot be regarded as the sole or decisive factor. In fact, the relative intensity of the diffraction peaks in the X-ray powder diffraction pattern is related to the preferred orientation of the crystals, and the diffraction peak intensities shown herein are illustrative and absolute comparison is not required. In addition, the experimental error of the diffraction peak position is usually 5% or less, and the error of these positions should also be taken into account. An error of ±0.2° is usually allowed. In addition, experimental factors such as sample thickness may cause the overall offset of the diffraction peak, and a certain offset is usually allowed. Thus, it will be understood by those skilled in the art that a crystalline form of the present disclosure is not necessarily to have the exactly same X-ray diffraction pattern of the example shown herein and the “same X-ray powder diffraction pattern” as described herein is not meaning absolutely the same, the same peak position can differ by ±0.2° and the peak intensity allows for some variability. Any crystalline forms whose X-ray diffraction patterns have the same or similar characteristic peaks should be within the scope of the present disclosure. Those skilled in the art can compare the patterns shown in the present disclosure with that of an unknown crystalline form in order to identify whether these two groups of patterns reflect the same or different crystalline forms.

In some embodiments, crystalline Form J of the present disclosure are pure, single, and substantially free of any other crystalline forms. In the present disclosure, when used to describe a novel crystalline form, the term “substantially free” means that the content of other crystalline forms in the novel crystalline form is less than 20% (w/w), specifically less than 10% (w/w), more specifically less than 5% (w/w) and further more specifically less than 1% (w/w).

It should be noted that a numerical value and a numerical range in the present disclosure should not be narrowly understood as the numerical value itself or the numerical range itself. It should be understood by those skilled in the art that the specific numerical value can be floated according to the specific technical environment on the basis of not departing from the spirit and principle of the present disclosure. In the present disclosure, the number of floating ranges which can be expected by one of skilled in the art is represented by the term “about”. The upper limit value and the lower limit value of the numerical range described in the specification of the present disclosure can be arbitrarily combined.

Compared with the prior art, crystalline Form J of the compound of formula (I) provided by the present disclosure has the advantages in at least one of solubility, melting point, stability, dissolution, hygroscopicity, adhesion, fluidity, biological effectiveness, processing performance, purification, formulation production, safety, and the like, which provides a new and better choice for the preparation of pharmaceutical formulations containing the compound of formula (I), and has a very important significance for the drug development.

### BRIEF DESCRIPTION OF THE DRAWINGS

FIG. 1 illustrates the XRPD pattern of Form J of Example 1.

FIG. 2 illustrates the TGA curve of Form J of Example 1.

FIG. 3 illustrates the DSC curve of Form J of Example 1.

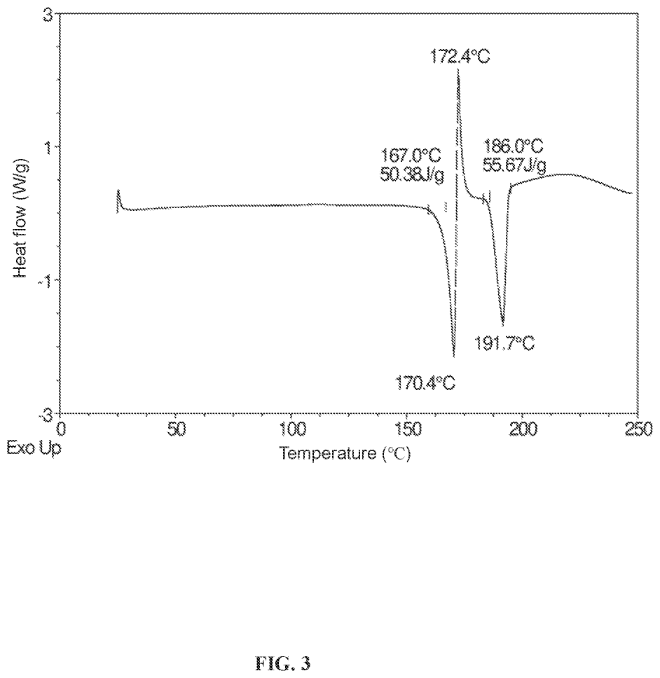

FIG. 4 illustrates ¹HNMR spectrum of Form J of Example 1.

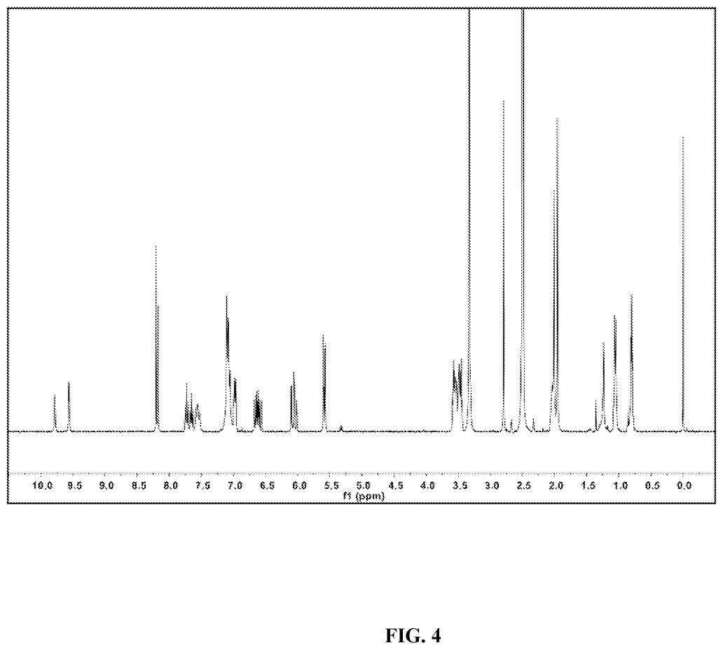

FIG. 5 illustrates the XRPD pattern of amorphous of Example 2.

FIG. 6 illustrates the DVS plot of Form A of Example 3.

FIG. 7 illustrates XRPD overlay of Form A of Example 3 before and after DVS test.

FIG. 8 illustrates the DVS plot of Form J of Example 1.

FIG. 9 illustrates the XRPD overlay of Form J of Example 3 before and after DVS test.

FIG. 10 illustrates the DVS plot of amorphous of Example 2.

FIG. 11 illustrates XRPD overlay of amorphous of Example 3 before and after DVS test.

FIG. 12 illustrates the solubility profile of different crystal forms in SGF.

FIG. 13 illustrates the solubility profile of different crystal forms in FaSSIF.

FIG. 14 illustrates the solubility profile of different crystal forms in FeSSIF.

FIG. 15 illustrates the solubility profile of different crystal forms in pure water.

FIG. 16 illustrates the dissolution profile of different crystal forms.

FIG. 17 illustrates the XRPD overlay of Form J under 25° C./60% RH.

FIG. 18 illustrates the XRPD overlay of Form J under 40° C./75% RH.

### DETAILED DESCRIPTION

### Form J

A J-type crystal of a compound, represented by formula (I), N-(3-(6-amino-5-(2-(N-methylacrylamido)ethoxy)pyrimidin-4-yl)-5-fluoro-2-methylphenyl)-4-cyclopropyl-2-fluorobenzamide, i.e., crystalline Form J (also referred to as Form J). By using the Cu—Kα radiation, Form J has an X-ray powder diffraction pattern comprising characteristic peaks at the 2θ values of 6.0°±0.2°, 6.8°±0.2°, and 20.6°±0.2°.

In an embodiment of the present disclosure, Form J has an X-ray powder diffraction pattern comprising characteristic peak(s) at one, two or three 2θ values of 12.2°±0.2°, 13.7°±0.2°, and 25.8°±0.2°.

In an embodiment of the present disclosure, Form J has an X-ray powder diffraction pattern comprising characteristic peak(s) at 2θ values of 12.2°±0.2°, 13.7°±0.2°, and 25.8°±0.2°.

In an embodiment of the present disclosure, Form J has an X-ray powder diffraction pattern comprising characteristic peak(s) at one, two or three 2θ values of 18.0°±0.2°, 21.0°±0.2°, and 27.6°±0.2°.

In an embodiment of the present disclosure, Form J has an X-ray powder diffraction pattern comprising characteristic peak(s) at 2θ values of 18.0°±0.2°, 21.0°±0.2°, and 27.6°±0.2°.

In an embodiment of the present disclosure, Form J has an X-ray powder diffraction pattern comprising characteristic peak(s) at four, five, six, seven, eight, or nine 2θ values of 6.0°±0.2°, 6.8°±0.2°, 12.2°±0.2°, 13.7°±0.2°, 18.0°±0.2°, 20.6°±0.2°, 21.0°±0.2°, 25.8°±0.2°, and 27.6°±0.2°.

In an embodiment of the present disclosure, Form J has an X-ray powder diffraction pattern comprising characteristic peak(s) at 2θ values of 6.0°±0.2°, 6.8°±0.2°, 12.2°±0.2°, 13.7°±0.2°, 18.0°±0.2°, 20.6°±0.2°, 21.0°±0.2°, 25.8°±0.2°, and 27.6°±0.2°.

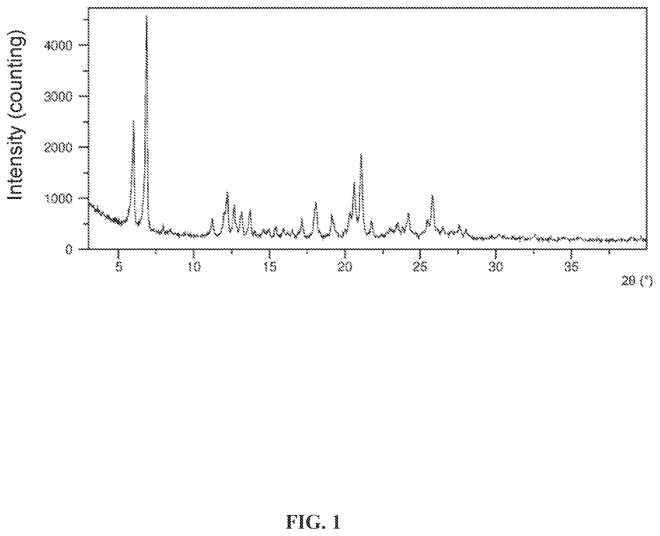

In an embodiment of the present disclosure, Form J has an X-ray powder diffraction pattern as shown in FIG. 1.

A process for preparing Form J includes dissolving the compound of formula (I) in an organic solvent, and obtaining Form J by evaporating a filtrate obtained after filtration. The evaporation was performed at RT.

According to the process for preparing Form J, the organic solvent was dibromethane.

### Amorphous

Amorphous of a compound, represented by formula (I), N-(3-(6-amino-5-(2-(N-methylacrylamido)ethoxy)pyrimidin-4-yl)-5-fluoro-2-methylphenyl)-4-cyclopropyl-2-fluorobenzamide is a solid.

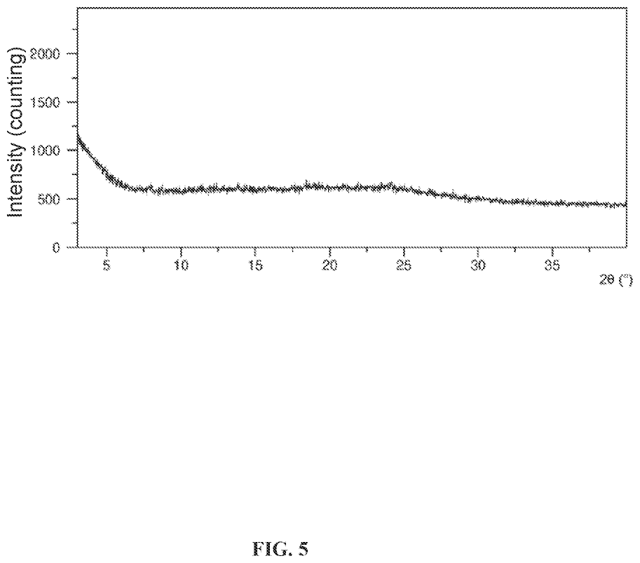

In an embodiment of the present disclosure, amorphous has an X-ray powder diffraction pattern as shown in FIG. 5.

A process for preparing the amorphous includes dissolving the compound of formula (I) in a mixed solvent; obtaining a clear filtrate by vacuum-concentrating the filtrate after filtration under a heating condition; performing reduced pressure distillation on the clear filtrate under a heating condition to remove most of the solvent; and obtaining a foamy-like solid by processing the mixture in the previous step using a vacuum oil pump under a heating condition.

In an embodiment of the present disclosure, the mixed solvent was dichloromethane and methanol.

In an embodiment of the present disclosure, the heating condition is 30° C. to 50° C.

The present disclosure will be further illustrated through following specific examples, which are not intended to limit the protection scope of the present disclosure. Those skilled in the art can make improvements to the preparation processes and the used instruments within the scope of the claims and specification, and those improvements should be considered as falling into the protection scope of the present disclosure. Therefore, the protective scope of the present disclosure should be defined by the appended claims.

In the present disclosure, “room temperature” generally refers to 22° C. to 28° C., unless otherwise specified.

The abbreviations used in the present disclosure are explained as follows:

XRPD: X-ray Powder Diffraction

DSC: Differential Scanning calorimetry analysis

TGA: ThermoGravimetric Analysis

¹H NMR: Nuclear Magnetic Resonance Hydrogen spectrum

DVS: Dynamic Vapor Sorption

PSD: Particle Size Distribution

PLM: Polarized Light Microscope

HPLC: High Performance Liquid Chromatography.

X-ray powder diffraction patterns of the present disclosure were collected on

Empyrean-Type and X′ Pert³-Type X-ray powder diffractometers of Panalytical Corporation. The process parameters of X-ray powder diffraction of the present disclosure were as follows:

X-ray source: Cu, Kα

Kα1 (Å): 1.54060; Kα2 (Å): 1.54443

Kα2/Kα1 intensity ratio: 0.50

Voltage: 45 kilovolts (kV)

Current: 40 milliamps (mA)

Scanning range: from 3.0 to 40.0 degrees (2θ angle).

Differential scanning calorimetry analysis charts of the present disclosure were collected on the Q200-type and Discovery DSC 2500-type differential scanning calorimeters of TA Company. The process parameters of the differential scanning calorimetry analysis of the present disclosure were as follows:

Scanning rate: 10° C./minute

Protection gas: Nitrogen gas.

The thermogravimetric analysis charts of the present disclosure were collected on Discovery TGA 5500-type and Q5000-type thermogravimetric analyzers of TA Company. The process parameters of the thermogravimetric analysis of the present disclosure were as follows:

Scanning rate: 10° C./minute

Protection gas: Nitrogen gas.

Nuclear magnetic resonance hydrogen spectrum data CH NMR) of the present disclosure were collected from a Bruker Avance II DMX 400M HZ nuclear magnetic resonance spectrometer. 1-5 mg of a sample was weighed, and dissolved in 0.5 mL of deuterated dimethyl sulfoxide to formulate 2-10 mg/mL of a solution for test.

The dynamic vapor sorption diagrams of the present disclosure were collected on Intrinsic-type and Intrinsic Plus-type dynamic vapor sorption instruments of SMS company. The process parameters of the dynamic vapor sorption test of the present disclosure were as follows:

Temperature: 25° C.

Protection gas and its flow rate: N₂, 200 mL/minute

dm/dt: 0.002%/minute

Minimum dm/dt equilibration time: 10 minutes

Maximum Equilibration Time: 180 minutes

Relative humidity range: 0% RH-95% RH-0% RH, 50% RH-95% RH-0% RH

Relative humidity gradient: 10% (0% RH-90% RH-0% RH), 5% (90% RH-95% RH and 95% RH-90% RH).

The raw material of formula (I) in the invention could be obtained via commercial purchase.

### Example 1: Preparation of Form J

Weigh appropriate amount of formula (I) solid into a 3-mL vial at RT, and then add in 3.0 mL of dibromomethane to dissolve the solid. Filter the mixture into a new 5-mL vial using 0.45 μm PTFE filter membrane. Seal the vial with parafilm and then prick 4 pinholes. Place the vial at RT for slow evaporation.

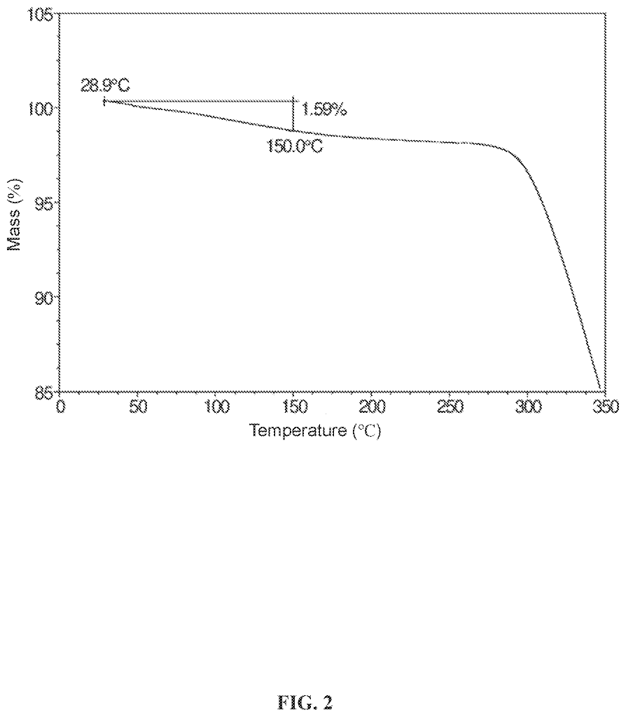

The obtained solid was determined to be Form J after characterization. The XRPD diagram of Form J was shown in FIG. 1, and the corresponding peak diffraction data was summarized in Table 1. Form J has characteristic reflection peaks at 2 theta values of 6.0°±0.2°, 6.8°±0.2°, 12.2°±0.2°, 13.7°±0.2°, 17.1°±0.2°, 18.0°±0.2°, 20.6°±0.2°, 21.0°±0.2°, 21.7°±0.2°, 25.8°±0.2°, and 27.6°±0.2°. The TGA, DSC and ¹H NMR of Form J was shown in FIG. 2˜4, respectively. TGA result showed a weight loss of 1.6% up to 150° C., DSC result showed that melting temperature of Form J was 167° C. (onset temperature).

TABLE 1

| Pos. [°2θ] | d-spacing [Å] | Rel. Int. [%] |
|---|---|---|
| 5.95 | 14.86 | 45.23 |
| 6.82 | 12.96 | 100.00 |
| 11.16 | 7.93 | 1.05 |
| 12.17 | 7.27 | 7.51 |
| 12.60 | 7.02 | 7.07 |
| 13.12 | 6.75 | 5.75 |
| 13.66 | 6.48 | 12.12 |
| 15.37 | 5.77 | 1.18 |
| 17.10 | 5.19 | 2.85 |
| 18.04 | 4.92 | 5.32 |
| 19.13 | 4.64 | 2.98 |
| 20.56 | 4.32 | 21.90 |
| 21.04 | 4.22 | 19.07 |
| 21.70 | 4.10 | 3.86 |
| 23.40 | 3.80 | 1.93 |
| 23.75 | 3.74 | 1.83 |
| 24.15 | 3.68 | 3.19 |
| 25.40 | 3.50 | 2.79 |
| 25.75 | 3.46 | 12.14 |
| 26.42 | 3.37 | 2.75 |
| 26.95 | 3.31 | 1.85 |
| 27.55 | 3.24 | 4.54 |
| 32.51 | 2.75 | 0.96 |

### Example 2: Preparation of Amorphous

Weigh appropriate amount of formula (I) solid into a 20-mL glass vial, and then add in 4.0 mL DCM and 14.0 mL MeOH to dissolve the solid. Filter the mixture into a new 50-mL vial using 0.45 pm PTFE filter membrane. Vacuum evaporate the filtrate at 45° C. to remove most of solvent. Vacuum dry the sample at 40° C. to obtain a foam-like solid.

The obtained solid was determined to be amorphous after characterization, and the corresponding XRPD diagram was shown in FIG. 5.

### Example 3: Hygroscopicity

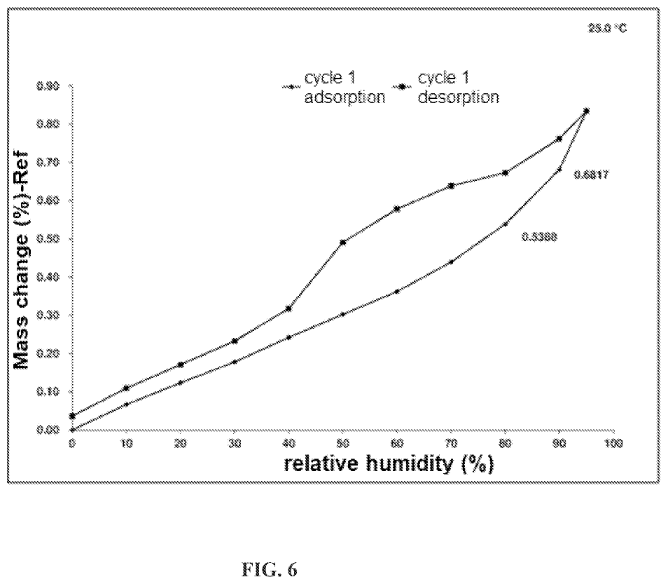

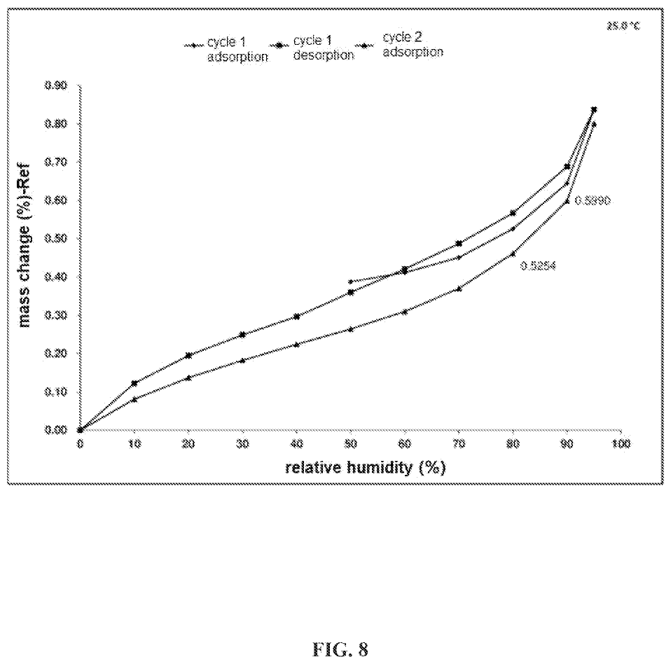

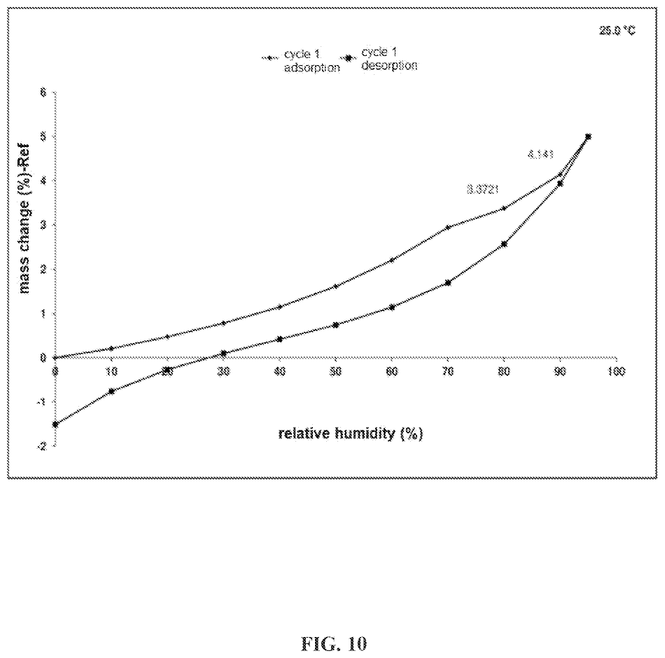

Weigh approximately 10 mg of a form of formula (I) (i.e., Form A from WO2020234779A1), 10 mg of Form J and 10 mg of amorphous for DVS test, and then collect the obtained solid for XRPD analysis. The DVS plots of Form A, Form J and amorphous were shown in FIGS. 6, 8 and 10, and the XRPD overlay were shown in

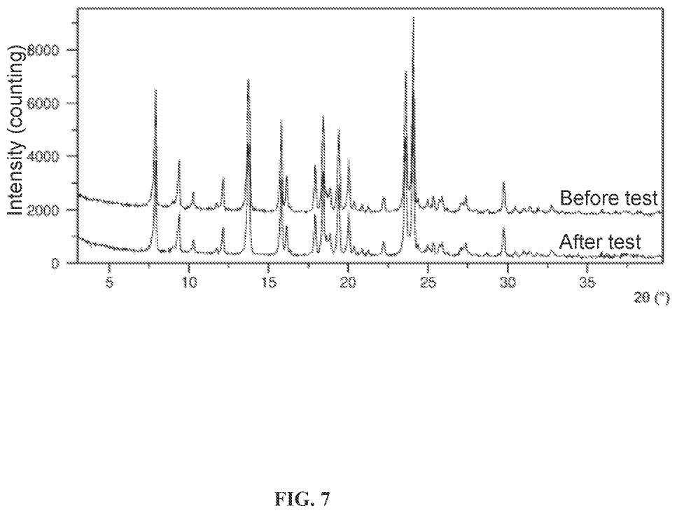

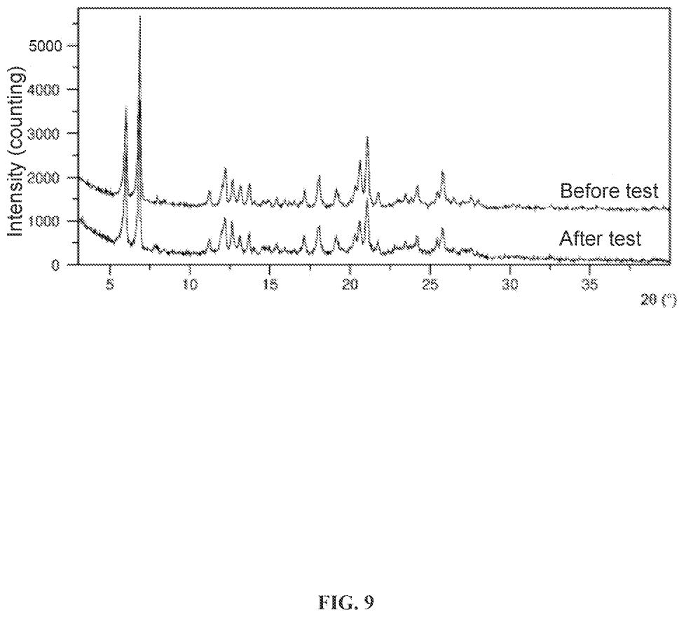

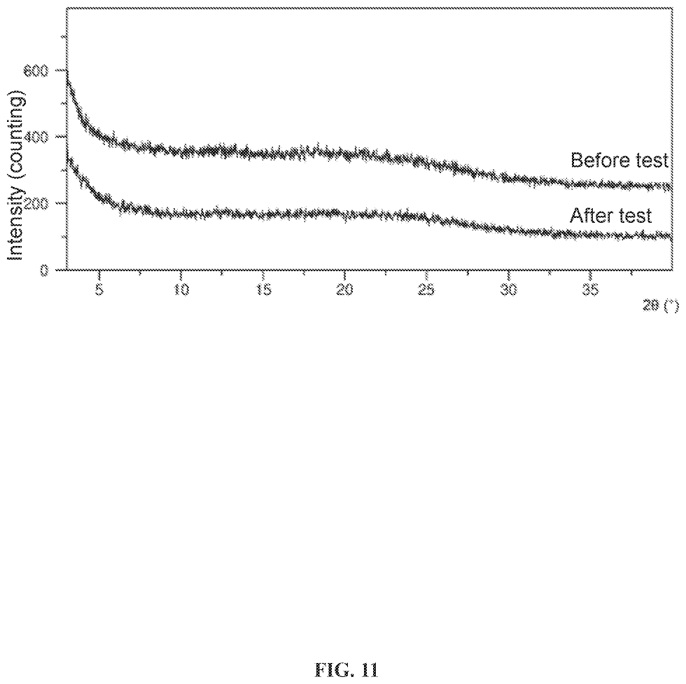

FIGS. 7, 9 and 11. The results were summarized in Table 2.

TABLE 2

| | Water uptake | Water uptake | | |
|---|---|---|---|---|
| Solid form | at 25° C., 80% | at 25° C., 90% | Hygroscopicity | Form change |
| Form A | 0.54% | 0.68% | Slightly | No form |
| | | | hygroscopic | change |
| Form J | 0.53% | 0.60% | Slightly | No form |
| | | | hygroscopic | change |
| Amorphous | 3.37% | 4.14% | Hygroscopic | No form |
| | | | | change |

According to “Pharmacopoeia of the People's Republic of China 2010” on the definition of hygroscopicity, the water uptake of Form J between 80% relative humidity and 90% relative humidity is slightly lower than that of Form A, but significantly lower than that of amorphous. Form J showed a water uptake of 0.60% at 90% relative humidity, indicating Form J is slightly hygroscopic. Also, Form J was relatively stable under high humidity condition, demonstrating Form J would keep stable in the process of production, storage and use, without specific humidity control conditions, which could well meet the requirements of pharmaceutical production and use.

Following is Description and definition of hygroscopicity (see appendix XIX J, guidance for hygroscopicity evaluation of Pharmacopoeia of the People's Republic of China 2010):

Deliquescent: sufficient water is absorbed to form a liquid,

Very hygroscopic: increase in mass is equal to or greater than 15 percent,

Hygroscopic: increase in mass is less than 15 percent and equal to or greater than 2 percent,

Slightly hygroscopic: increase in mass is less than 2 percent and equal to or greater than 0.2 percent.

Non-hygroscopic: increase in mass is less than 0.2 percent.

### Example 4: Kinetic Solubility

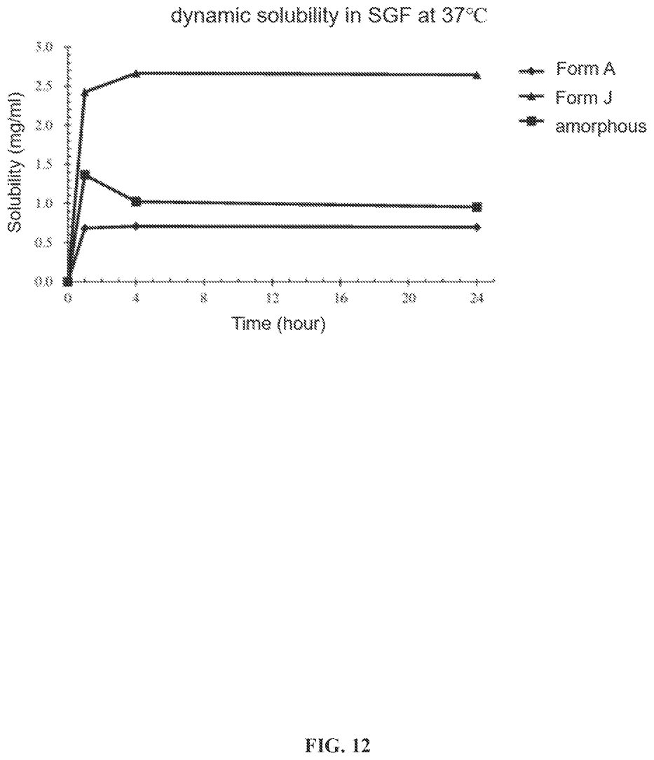

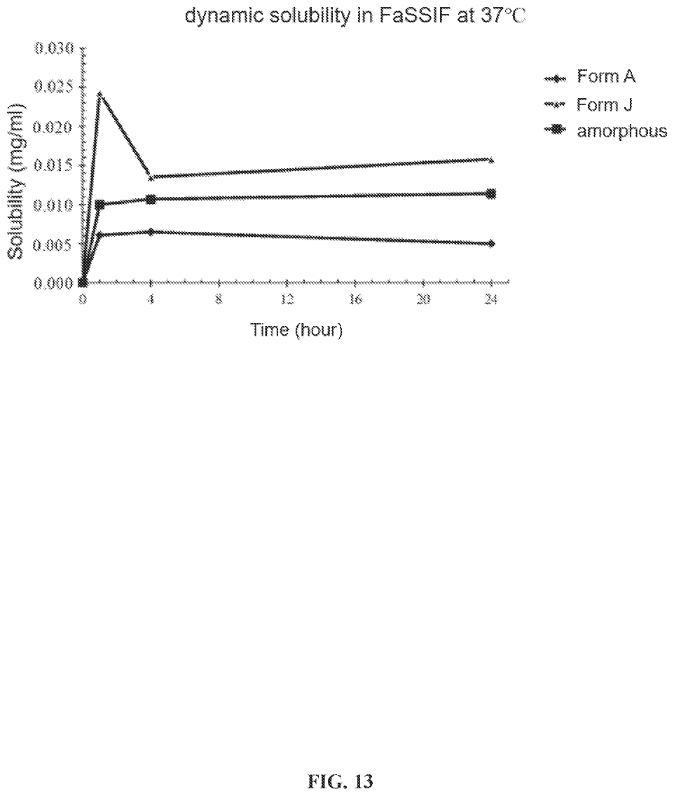

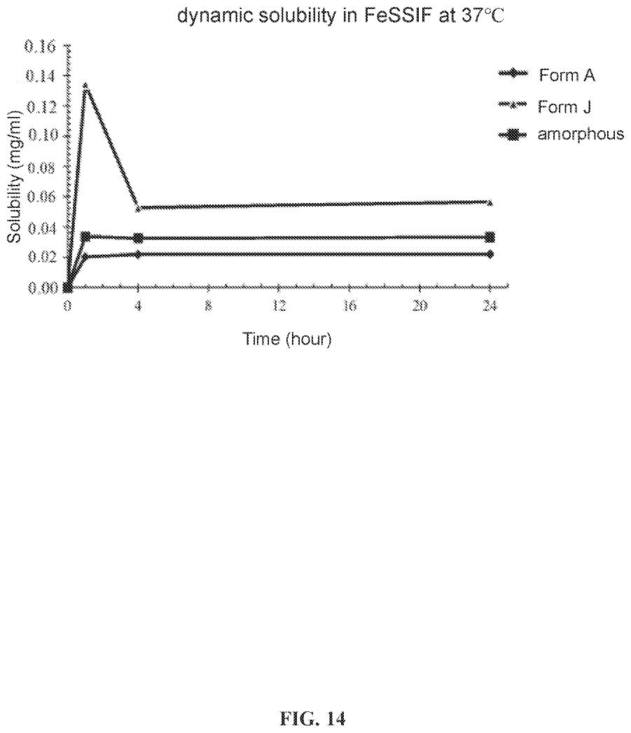

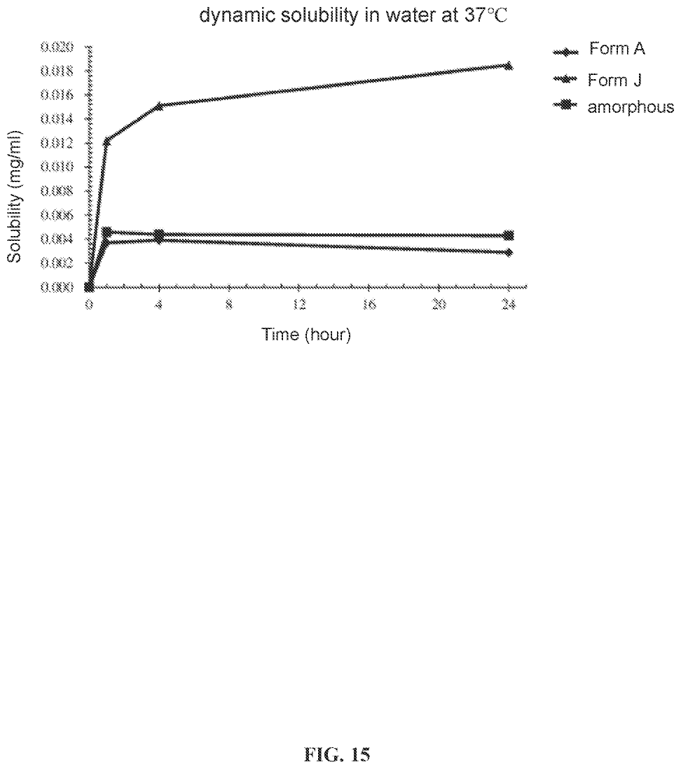

Suspend Form J (from example 1), amorphous (from example 2) and Form A (from WO2020234779A1) in Simulated Gastric Fluid (SGF), Fasted-State Simulated Intestinal Fluid (FaSSIF), Fed-State Simulated Intestinal Fluid (FeSSIF) and pure water, respectively. After equilibrium for 1 hour, 8 hours and 24 hours, filter the solution for HPLC test. The results were summarized in Table 3, and solubility profiles were shown in FIGS. 12 to 15. As a result, Form J showed a higher solubility than amorphous or Form A in all medias.

TABLE 3

| | | | |
|---|---|---|---|
| Media | 1 hr(mg/mL) | 4 hr (mg/mL) | 24 hr (mg/mL) |

Form A

| | | | |
|---|---|---|---|
| SGF | 0.69 | 0.71 | 0.70 |
| FaSSIF | 0.0061 | 0.0065 | 0.0050 |
| FeSSIF | 0.020 | 0.022 | 0.022 |
| H₂O | 0.0037 | 0.0039 | 0.0029 |

Amorphous

| | | | |
|---|---|---|---|
| SGF | 1.4 | 1.0 | 0.95 |
| FaSSIF | 0.010 | 0.011 | 0.011 |
| FeSSIF | 0.033 | 0.034 | 0.033 |
| H₂O | 0.0046 | 0.0044 | 0.0043 |

Form J

| | | | |
|---|---|---|---|
| SGF | 2.4 | 2.7 | 2.6 |
| FaSSIF | 0.024 | 0.014 | 0.016 |
| FeSSIF | 0.13 | 0.053 | 0.057 |
| H₂O | 0.012 | 0.015 | 0.019 |

### Example 5: Dissolution

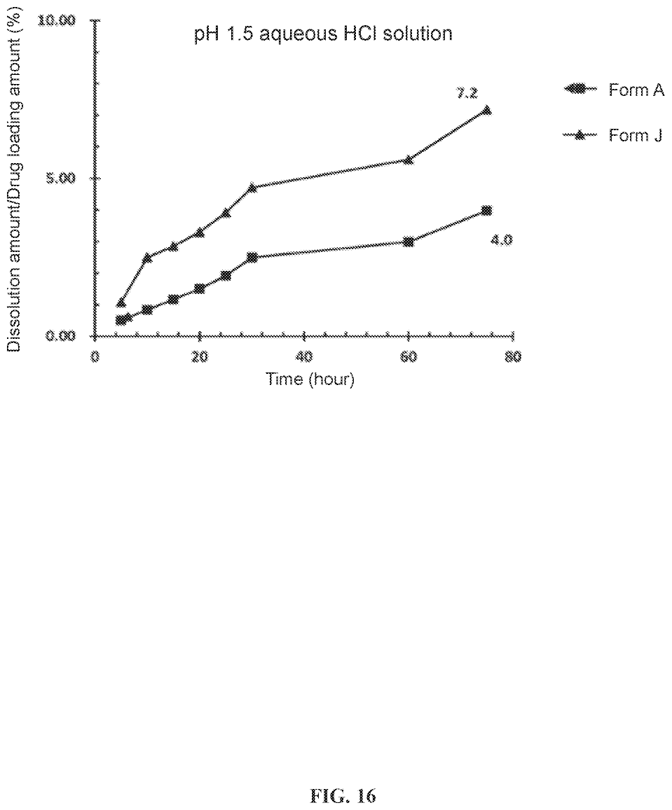

Approximately 100 mg of each of Form J (from example 1) and Form A (from WO2020234779A1) were compressed by intrinsic dissolution mold under 10 kN pressure to obtain a thin tablet with surface area of 1.0 cm2, respectively. Dissolution experiment was performed on the tablets, and the corresponding result was summarized in Table 4, dissolution profile was shown in FIG. 16. As a result, Form J showed larger total dissolution amount and higher cumulative dissolution rate than Form A.

TABLE 4
pH 1.5 aqueous HCl solution

Cumulative dissolution amount (mg)

| Time (mins) | Form A | Form J |
|---|---|---|
| 5 | 0.5 | 1.1 |
| 10 | 0.9 | 2.5 |
| 15 | 1.3 | 2.9 |
| 20 | 1.6 | 3.3 |
| 25 | 2.1 | 3.9 |
| 30 | 2.7 | 4.7 |
| 60 | 3.2 | 5.6 |
| 75 | 4.3 | 7.2 |

### Example 6: Solid-State Stability of Form J

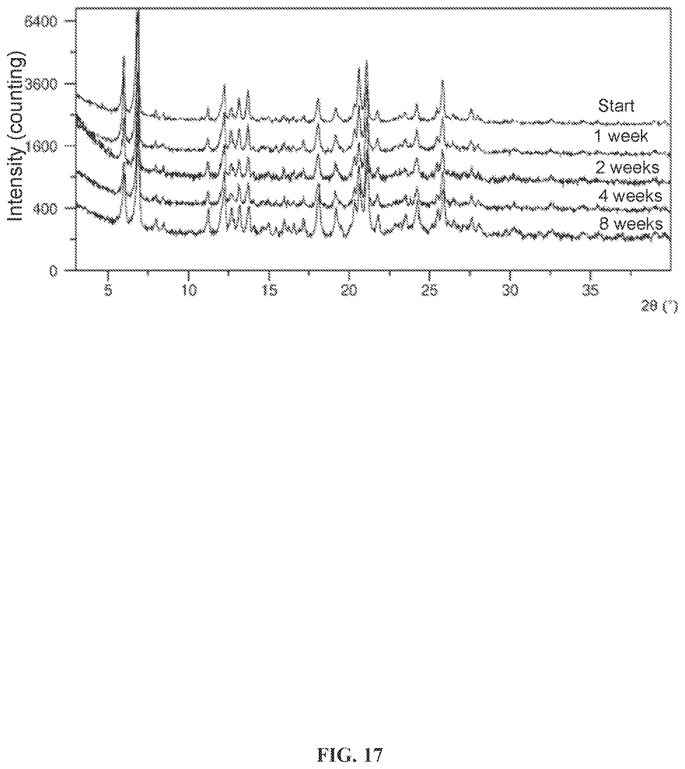

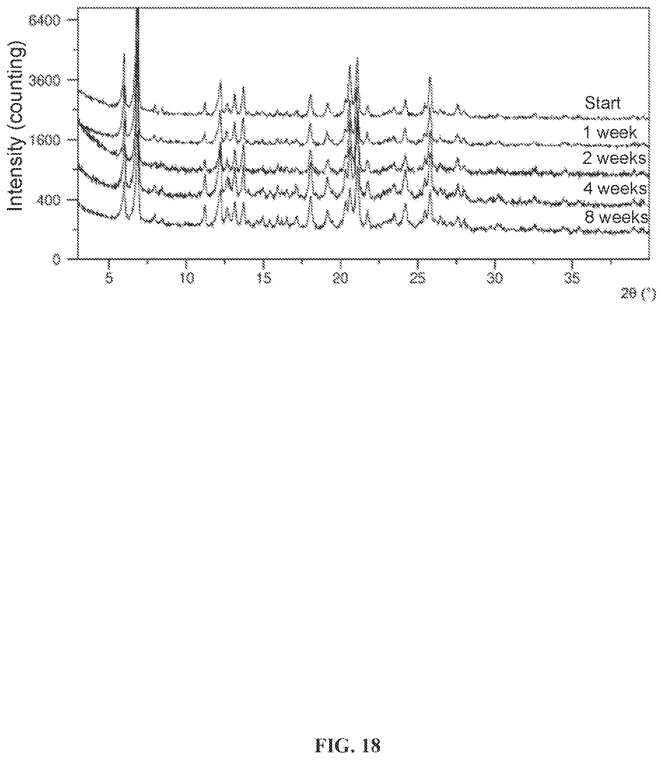

Weigh appropriate amount of Form J (from example 1) into a 5-mL glass vial, and then seal the vial with parafilm (pricked 5 pinholes). Place the vial under 25° C./60% RH and 40° C./75% RH for some time, and then separate several solids out for XRPD and HPLC purity test. The results were summarized in Table 5, and the XRPD overlay was shown in FIGS. 17 to 18. As a result, no obvious form change and HPLC purity decrease was observed after keeping Form J under 25° C./60% RH and 40° C./75% RH for 8 weeks, indicating Form J showed a good stability and meets the requirement of pharmaceutical production and use for stability.

TABLE 5

| | | | Time | | Purity | Purity/initial |
|---|---|---|---|---|---|---|
| | Mass | | point | Solid | (area | Purity |
| Entry | (mg) | Condition | (weeks) | form | %) | (%) |
| 1 | 5.85 | 25° C./ | 1 | Form J | 99.09 | 99.6 |
| 2 | 6.52 | 60% RH | 2 | Form J | 98.48 | 99.0 |
| 3 | 5.88 | | 4 | Form J | 98.71 | 99.2 |
| 4 | 10.84 | | 8 | Form J | 97.94 | 98.7 |
| 5 | 6.26 | 40° C./ | 1 | Form J | 99.05 | 99.6 |
| 6 | 5.19 | 75% RH | 2 | Form J | 98.39 | 98.9 |
| 7 | 6.26 | | 4 | Form J | 98.42 | 99.0 |
| 8 | 10.89 | | 8 | Form J | 97.32 | 98.0 |

### Example 7: Crystal Habit

About 10 mg of Form J (from example 1) and Form A (from WO2020234779A1) were weighed and placed onto a glass slide, respectively, the solid were dispersed with several vacuum pump oil, and then covered with a cover glass. The samples were observed using PLM. As a result, Form J showed better crystal habit than Form A.

### Example 8: PSD Test

Approximately 10-30 mg of each of Form J (from example 1) and Form A (from WO2020234779A1) were added into 5 mL Isopar G (0.2% lecithin) to form a suspension, respectively. The suspension was loaded into SDC system for data collection, which was collected before and after sonication for 30 s. As a result, Form J showed more uniform particle size distribution than Form A.

### Example 9: Sticking Propensity

Approximately 30 mg of each of Form J (from example 1) and Form A (from WO2020234779A1) were compressed by 6 mm circle punches under 10 kN pressure, respectively. The loading mass (ML) and tablet mass (MT) were recorded, and mass percentage of powder adhering to punch tip (ma) was calculated by the equation of ma=(ML−MT)/ML×100%. As a result, Form J showed lower sticking propensity than Form A.

The above-mentioned embodiments are only intended to illustrate the technical concept and characteristics of the present disclosure, and the purpose thereof is to enable those who are familiar with the art to understand the content of the present disclosure and implement them accordingly, and cannot limit the protection scope of the present disclosure. All equivalent changes or modifications made according to the spirit of the present disclosure should be included within the protection scope of the present disclosure.

1. A crystalline Form J of a compound, represented by formula (I), with the chemical name of N-(3-(6-amino-5(2-(N-methacrylamide)ethoxy)pyrimidin-4-yl)-5-fluoro-2-methylphenyl)-4-cyclopropyl-2-fluorobenzamide, wherein an X-ray powder diffraction pattern of the crystalline Form J obtained by using Cu—Kα radiation, has characteristic peaks at 2θ values of 6.00±0.20, 6.8°±0.2°, and 20.6°±0.2°.

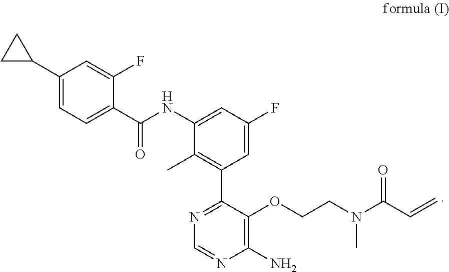 [SMILES: C=CC(=O)N(C)CCOc1c(N)ncnc1-c1cc(F)cc(NC(=O)c2ccc(C3CC3)cc2F)c1C]

2. The crystalline Form J of claim 1, wherein the X-ray powder diffraction pattern has one, two, or three characteristic peaks at 2θ values of 12.2°±0.2°, 13.7°±0.2°, and 25.8°±0.2°.

3. The crystalline Form J of claim 1, wherein the X-ray powder diffraction pattern has characteristic peaks at 2θ values of 12.2°±0.2°, 13.7°±0.2°, and 25.8°±0.2°.

4-7. (canceled)

8. A method for preparing the crystalline Form J of claim 1, the method comprising:
(a) dissolving a sample including the compound represented by the formula (I) into an organic solvent to obtain a solution;
(b) filtering the solution to obtain a filtrate; and
(c) obtaining the crystalline Form J by evaporating the filtrate.

9. The method of claim8, wherein the organic solvent is dibromomethane.

10. The method of claim 8, wherein the solution is filtered by using a filter membrane.

11. The method of claim 8, wherein the filtrate is evaporated at room temperature.

12. A pharmaceutical composition, comprising the crystalline Form J of claim 1 and a pharmaceutically acceptable carrier.

13. The pharmaceutical composition of claim 12, wherein the pharmaceutical composition has a Bruton's tyrosine kinase (BTK) inhibitory activity.

14. The pharmaceutical composition of claim 12, wherein the crystalline Form J is used as an active ingredient.

15. A method for preventing chronic urticaria, asthma, or Sjogren's syndrome, the method comprising: administering an effective amount of the crystalline Form J of claim 1 to a subject.

16. A method for treating chronic urticaria, asthma, or Sjogren's syndrome, the method comprising: administering an effective amount of the crystalline Form J of claim 1 to a subject suffering from urticaria, asthma, or Sjogren's syndrome.
